(19) United States
(12) Patent Application Publication
(10) Pub. No.: US 2025/0160669 A1
(43) Pub. Date: May 22, 2025

(54) Photoplethysmogram Sensor with Skin-Tone Compensation

(71) Applicant: Trustees of Tufts College, Medford, MA (US)

(72) Inventors: Valencia Joyner Koomson, Danvers, MA (US); Ravi Durbha, Somerville, MA (US)

(21) Appl. No.: 19/029,757

(22) Filed: Jan. 17, 2025

Related U.S. Application Data
(63) Continuation-in-part of application No. PCT/US2023/069927, filed on Jul. 11, 2023.
Application No. PCT/US2023/069927 is a continuation of application No. 18/184,468, filed on Mar. 15, 2023, now abandoned.
(60) Provisional application No. 63/390,782, filed on Jul. 20, 2022.

(51) Int. Cl. A61B 5/0295; A61B 5/026

(52) U.S. Cl. CPC A61B 5/0295; A61B 5/0261

(57) ABSTRACT

An improved photoplethysmogram sensor for measuring a set of parameters related to changes in arterial blood volume in tissue of a subject has a set of compensation LEDs configured to illuminate the tissue in the course of a compensation process; a set of compensation photodetectors configured to measure light, returned by the tissue from the set of compensation LEDs during the compensation process, to provide as an output a signal indicative of the spectral response of the tissue; and a control circuit configured to use the output from the set of photodetectors to compensate for the spectral response of the tissue in a manner to reduce errors associated with subject-to-subject variation in light transmissivity of tissue.

### RELATED APPLICATIONS

This application is a continuation-in-part of international application serial number PCT/US2023/069927, filed Jul. 11, 2023, which claims priority to U.S. application Ser. No. 18/184,468, filed Mar. 15, 2023, which claims the benefit of U.S. provisional application Ser. No. 63/390,782, filed Jul. 20, 2022. Each of the foregoing applications is hereby incorporated herein by reference in its entirety.

### GOVERNMENT FUNDING

The present invention was made under National Science Foundation Grant number 1919038 and is subject to rights in the National Science Foundation.

### TECHNICAL FIELD

The present invention relates to pulse oximeters, and more particularly to pulse oximeters having color compensation.

### BACKGROUND ART

Photoplethysmogram sensors, which measure blood volume changes in the microvascular system, and are frequently implemented as pulse oximeters to measure blood-oxygen saturation, are well known in the art. Representative pulse oximeters are described in U.S. Pat. Nos. 6,879,850, 6,912,413, and 4,859,057, and the prior art described therein is incorporated in this section by reference.

A team of physicians from Hypoxia Lab, University of California, San Francisco (UCSF) studied performance of multiple pulse oximeters for accuracy in measuring saturation of peripheral oxygen (SpO₂) for over eighteen years. In 2005 it was concluded that the accuracy of pulse oximeters is biased since they are calibrated with light-skinned individuals, with the assumption that skin pigment has no effect in measuring blood oxygenation. A follow-up study conducted in 2007 revealed that some pulse oximeters overestimate arterial oxyhemoglobin saturation levels in individuals with darkly pigmented skin. In 2020 similar results were reported by a team of doctors from University of Michigan Medical School.

### SUMMARY OF THE EMBODIMENTS

In accordance with one embodiment of the invention, there is provided an improved photoplethysmogram sensor for measuring a set of parameters related to changes in arterial blood volume in tissue of a subject, the photoplethysmogram sensor being of the type including first and second primary optical sources configured to transmit light into the tissue at selected red and infrared wavelengths respectively, a set of primary photodetectors to measure light returned by the tissue in response to such transmission, and a control circuit coupled to the light sources and the set of primary photodetectors. The improvement comprises:

a set of compensation optical sources configured to illuminate the tissue in the course of a compensation process; and

a set of compensation photodetectors configured to measure light, returned by the tissue from the set of compensation optical sources during the compensation process, provide an output signal indicative of the spectral response of the tissue;

wherein the control circuit is coupled to the set of compensation optical sources and to the set of photodetectors and is configured to use the output from the set of photodetectors to compute and to deliver an adjustment to power supplied to the first primary optical source in relation to power supplied to the second primary optical source to compensate for the spectral response of the tissue, in a manner to reduce errors associated with subject-to-subject variation in light transmissivity of tissue.

In a further related embodiment, the improvement further comprises an inertial measurement unit (IMU) coupled to the control circuit, and the control circuit is configured to compensate for artifacts attributable to motion of a set of components of the photoplethysmogram sensor.

In another further related embodiment, the control circuit is further configured to implement a calibration process, ahead of an actual measurement, in which the set of photodetectors monitors outputs of the first and second primary optical sources and is used by the control circuit to adjust these outputs to reduce the effects of spectral changes in outputs of the first and second primary optical sources.

Optionally, in various embodiments, the first and second primary optical sources may be implemented as LEDs. Similarly, the set of compensation optical sources may be implemented as a set of LEDs. Also optionally, the control circuit may be implemented as a microcontroller. In a further related embodiment, the set of compensation optical sources includes a white-light emitting light source. In another related embodiment, the set of compensation optical sources includes an RGB photodiode array. In a further related embodiment, the control circuit is configured to cause carrying out of the compensation process in a distinct compensation time slot of a measurement cycle. Optionally, the control circuit is configured to cause carrying out of a data acquisition process, during which the first and second primary optical sources and the primary light-sensitive semiconductors are operational in an acquisition time slot following the compensation time slot in the measurement cycle and the first and second primary optical sources are powered in a manner reflecting the adjustment.

In a further related embodiment, the control circuit is configured to use the output from the set of photodetectors to produce an objective measure of the spectral response of the tissue. Optionally, the control circuit is configured to use the output from the set of photodetectors to determine a value that is correlated with an Individual Typology Angle (ITA) of the tissue. As a further option, the control circuit is configured to determine the value correlated with the ITA based a set of parameters corresponding to coordinates under the CIE 1931 XYZ standard and thereafter to use such parameters to calculate values corresponding to CIELAB standards based on the CIE 1976 L*a*b* color space.

In another related embodiment, there is provided An improved photoplethysmogram sensor for measuring a set of parameters related to changes in arterial blood volume in tissue of a subject, the photoplethysmogram sensor being of the type including first and second primary optical sources configured to transmit light into the tissue at selected red and infrared wavelengths respectively, a set of primary light-sensitive semiconductors to measure light returned by the tissue in response to such transmission, and a control circuit coupled to the light sources and the set of primary light-sensitive semiconductors, wherein the improvement comprises:

a set of compensation optical sources configured to illuminate the tissue in the course of a compensation process;

a set of compensation photodetectors configured to measure light, returned by the tissue from the set of compensation optical sources during the compensation process, to provide as an output a signal indicative of the spectral response of the tissue; and

an inertial measurement unit (IMU) coupled to the control circuit;

wherein the control circuit is coupled to the set of compensation optical sources, and to the set of photodetectors, and to the IMU, and is configured (i) to use the output from the set of photodetectors to compute and to deliver an adjustment to power supplied to the first primary optical source in relation to power supplied to the second primary optical source to compensate for the spectral response of the tissue, in a manner to reduce errors associated with subject-to-subject variation in light transmissivity of tissue, (ii) to compensate for artifacts attributable to motion of a set of components of the photoplethysmogram sensor, and (iii) to implement a calibration process, ahead of an actual measurement, in which the set of photodetectors monitors outputs of the first and second primary optical sources and is used by the control circuit to adjust these outputs to reduce the effects of spectral changes in outputs of the first and second primary optical sources.

### BRIEF DESCRIPTION OF THE DRAWINGS

The foregoing features of embodiments will be more readily understood by reference to the following detailed description, taken with reference to the accompanying drawings, in which:

FIG. 1 is a side view of a color-compensated photoplethysmogram sensor in accordance with an embodiment of the present invention; and

FIG. 2 is a system block diagram of the color-compensated photoplethysmogram sensor of FIG. 1, in accordance with a further embodiment of the present invention.

FIG. 3 is a diagram of a measurement cycle implemented with time-division multiplexing of processes associated with operation of the embodiment of FIG. 2, in accordance with a further embodiment of the present invention.

FIG. 4 is a diagrammatic representation of the LAB Color Space according to the color organization system of the Commission Internationale de l′Elcairage (CIE, the International Commission on Illumination).

FIG. 5 is a graph showing how, a position in the CIE LAB Color Space of FIG. 4 can be mapped to values of L*, a*, and b* from which there can be computed the Individual Typology Angle (ITA).

FIG. 6 is a schematic diagram of an I2C programmable LED driver circuit used to power the RED and IR LEDs of an embodiment of the present invention.

### DETAILED DESCRIPTION OF SPECIFIC EMBODIMENTS

Definitions. As used in this description and the accompanying claims, the following terms shall have the meanings indicated, unless the context otherwise requires:

A “set” includes at least one member.

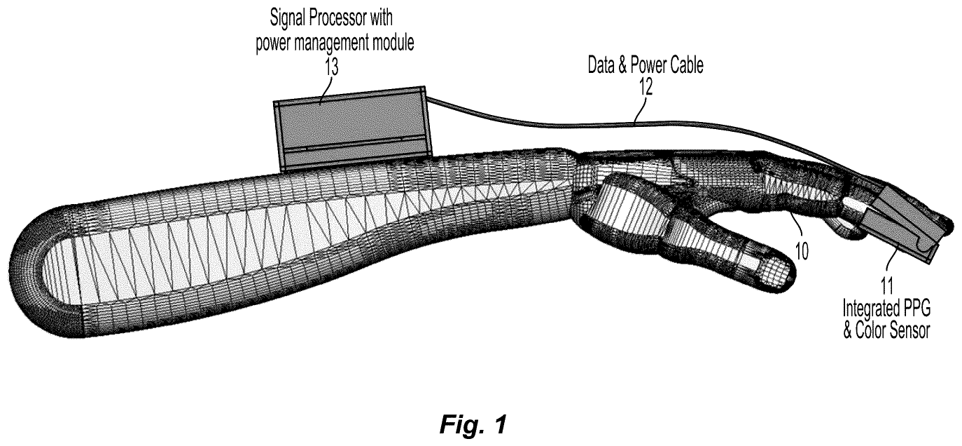

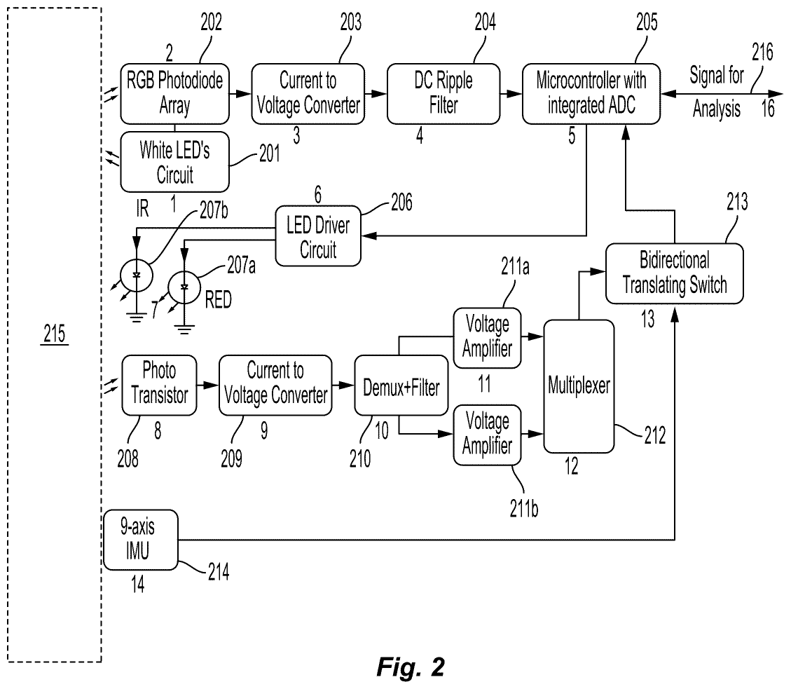

FIG. 1 is a side view of a color-compensated photoplethysmogram sensor, as worn by a subject being tested, in accordance with an embodiment of the present invention. The photoplethysmogram sensor of this embodiment includes a finger-mounted unit 11, in which are mounted a set of primary LED transmitters, operating at red (660 nm) and infrared (895 nm) wavelengths, and a primary phototransistor 208 (shown in FIG. 2) to receive light returned as a result of the action of the transmitters, as well as a white LED transmitter used to illuminate tissue in a compensation process and an RGB photodiode array configured to measure light, returned by the tissue from the white LED transmitter during the compensation process, to provide as an output a signal indicative of the spectral response of the tissue. The finger-mounted unit also includes an inertial measurement unit (IMU). These items in the finger-mounted unit 11 are coupled over data and power cable 12 to the signal processor and power management module 13.

FIG. 2 is a system block diagram of the color-compensated photoplethysmogram sensor of FIG. 1, in accordance with a further embodiment of the present invention. The finger of the subject, shown as item 215, is irradiated with light at red (660 nm) and infrared (895 nm) wavelengths by primary LEDs 207a and 207b respectively. The primary phototransistor 208 serves as a photodetector that receives light at these wavelengths for purposes of photoplethysmography. The primary LEDs are driven by driver circuit 206, which in turn is controlled by the microcontroller 205. The microcontroller 205 includes an integrated analog-to-digital converter.

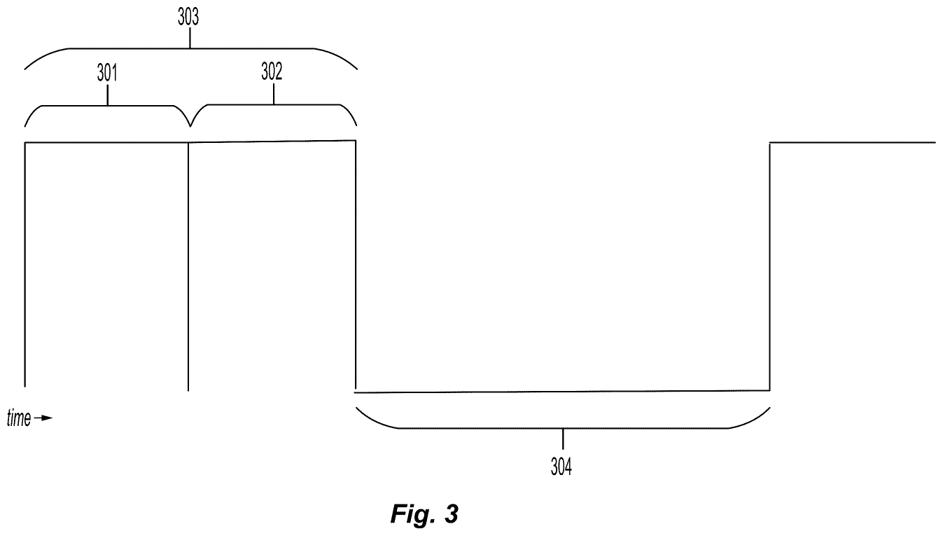

FIG. 3 is a diagram of a measurement cycle implemented with time-division multiplexing of processes associated with operation of the embodiment of FIG. 2, in accordance with a further embodiment of the present invention. In this context, we describe a novel arrangement to compensate for measurement inaccuracies introduced by subject-to-subject variations in skin tone and other sources of subject-to-subject variations in light transmissivity in tissue over the range of wavelengths employed for measurement of blood volume parameter changes. Time is indicated along the horizontal axis, and system activity is shown on the vertical axis. The system is powered on during frame 303 and powered off during frame 304 (when no data is gathered or recorded). Together frames 303 and 304 correspond to a complete period of operation of the system. When the system is powered on, in frame 303, there are two successive modes of operation, compensation (during time slot 301) and data acquisition (during time slot 302). During compensation mode 301, white LED circuit 201 (in FIG. 2), disposed in the finger-mounted unit 11, is powered on to transmit white light into the finger 215 of the subject. Also during compensation mode 301, the spectral response of the finger's tissue to this white illumination is captured by RGB photodiode array 202 to produce a color characterizing signal corresponding to the effects of skin tone of the subject and of other optical properties of the path traversed by light between the white LED of circuit 201 and the RGB photodiode array 202. The output of the RGB photodiode array 202 is processed in the current-to-voltage converter 203 and cleaned in DC ripple filter 204, before it is fed to the microcontroller 205, which uses the color characterizing signal to produce an objective measure of skin tone. That objective skin color measure is used in turn by the microprocessor to compute an adjustment to power supplied to the red LED 207a in relation to power supplied to the infrared LED 207b in the data acquisition time slot 302 to compensate for the spectral response of the subject's tissue measured in the compensation time slot 301.

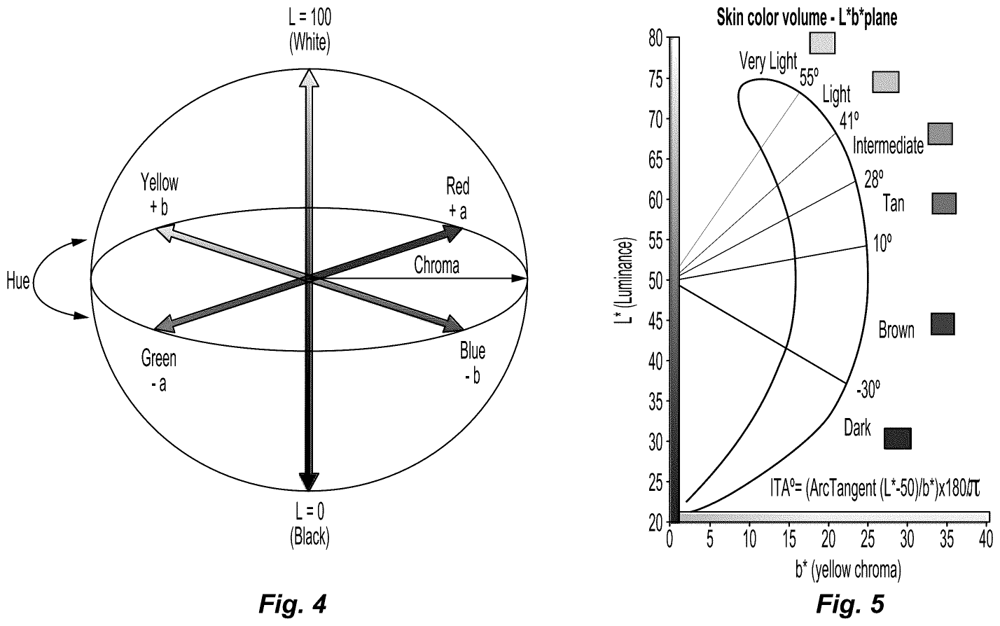

In various embodiments of the present invention, the objective measure of skin tone is determined in parameters corresponding to coordinates in a suitable system such as illustrated diagrammatically in FIG. 4, namely the LAB Color Space, according to the color organization system of the Commission Internationale de l′Elcairage (CIE, the International Commission on Illumination). See generally the CIE website at https://cie.co.at/node/2. An objective measure of skin tone is useful in characterizing physiological conditions such as skin cancer risk, variations in melanin composition, blood flow, photo aging and response to treatments.

FIG. 5 is a graph showing how a position in the CIE LAB Color Space of FIG. 4 can be mapped to values of L*, a*, and b* from which there can be computed the Individual Typology Angle (ITA), which is a measure of constitutive pigmentation of the skin based on colorimetric parameters. In various embodiments of the present invention, computing the Individual Typology Angle from the color characterizing signal produced by RGB photodiode array 202 involves a two-stage process. In the first stage, the color characterizing signal is converted into coordinates under the CIE 1931 XYZ standard (also known as the CIE 2° standard colorimetric system) where ‘X’ is a mix of three CIE RGB curves, ‘Y’ is the luminance and ‘Z’ is quasi-equal to blue. In the second stage, these XYZ values are converted to CIELAB standards based on CIE 1976 L*a*b* color space, where ‘L*’ is luminance, ‘a*’ is cutaneous erythema, and ‘b*’ is constitutional pigmentation and ability to acquire a tan. Individual Typology Angle (ITA) classifies skin tone into 7 physiological categories: ITA>55° very fair, 41°<ITA<55° fair, 28°<ITA<41° Intermediate, 10°<ITA<28° Tan, −30°<ITA<10° Brown, and ITA <−30° Dark complexion. ITA is computed by the formula:

$$ITA ° = \frac{arctan ( {L}^{*} - 50 )}{{b}^{*}} * \frac{180}{π}$$

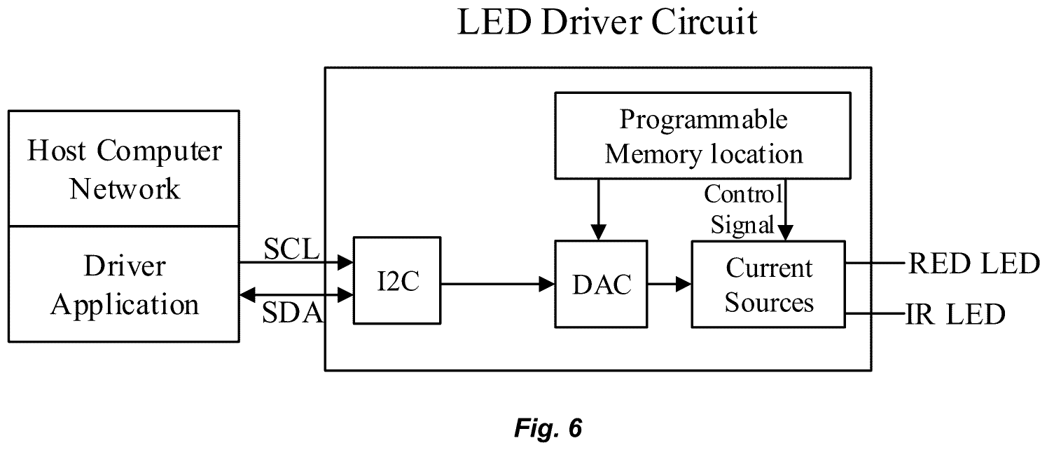

FIG. 6 is a schematic diagram of an I2C programmable LED driver circuit used to power the RED and IR LEDs of an embodiment of the present invention. The brightness of RED and IR LEDs is controlled by the I2C programmable LED driver circuit of FIG. 6 with a built in digital to analog conversion (DAC) circuit that maps the digital value input to an equivalent current value from the lookup table (LUT) programmed in memory location. The lookup table charts the current value in milliamps (mA) in relation to equivalent LED brightness values. The luminance value from the CIE 1976 L*a*b* standard has a range from 0 to 100, which is scaled to fit the min and max range of LED brightness. To The LED driver circuit is configured to generate current necessary to produce a luminance value that will minimize the influence of skin tone in computing vital signs accurately.

During the data acquisition time slot 302, power supplied to the red and infrared diodes 207a and 207b reflects the adjustment to power, described above in the context of the compensation time slot 301, to compensate for the spectral response of the subject's tissue. Data acquired during the acquisition time slot 302 is sampled at 100 Hz.

Although the phototransistor 208 produces an output that does not per se discriminate on the basis of the wavelength of light directed to it, the LEDs 207a and 207b are caused to be illuminated sequentially by the microcontroller 205, so that the output of phototransistor 208, after running though the current to voltage converter 209, is separated and filtered by a demultiplexer-filter (demux-filter) arrangement 210 into two distinctive outputs corresponding to the two different wavelengths of excitation. The outputs are amplified by voltage amplifiers 211a and 211b and then fed to multiplexer 212, which provides an input to bidirectional translating switch 213, and eventually a measure of the light reflected at the two wavelengths of illumination to the microcontroller 205.

Inertial Measurement Unit (IMU). The system also compensates for artifacts of motion of the finger-mounted unit by utilizing the inertial measurement unit 214, which is disposed in the finger-mounted unit 11 of FIG. 1. The inertial measurement unit 214 captures linear acceleration data in six directions (e.g., X, Y, and Z axes and their negative reflections) and angular acceleration about three axes, to provide a quantitative evaluation of motion with respect to each of nine distinct degrees of freedom. The output of the inertial measurement unit is another input to the bidirectional translating switch 213, the output of which is fed to the microcontroller 205, so that the microcontroller can compensate for the effects of motion of the finger-mounted unit 11. Bi-directional translational switch 213 helps to move motion artifact data along with photoplethysmogram (PPG) data to a remote computer via microcontroller 205 interface for signal analysis over line 216 to compute arterial blood oxygenation and pulse rate.

In various embodiments of the present invention, the IMU 214 is an advanced sensor module that combines a 3-axis accelerometer, a 3-axis gyroscope, and sometimes with a 3-axis magnetometer. This combination provides comprehensive data about an object's motion, orientation, and position in three-dimensional space. The accelerometer measures linear acceleration, the gyroscope captures rotational velocity, and the magnetometer detects the magnetic field, enabling precise heading and orientation estimation. By fusing data from these three sensors, a 9-axis IMU can accurately track motion and orientation, even in complex and dynamic environments. This makes it highly versatile for applications requiring precise motion sensing, such as robotics, navigation, gaming, and wearable devices.

Integrating an IMU with a pulse oximeter significantly enhances the device's ability to detect and compensate for motion artifacts, ensuring accurate and reliable readings of oxygen saturation (SpO2) and pulse rate, even during user movement. The IMU's accelerometer and gyroscope data help identify and characterize translational and rotational movements, while the magnetometer provides consistent orientation tracking, even in environments with varying magnetic or gravitational fields. By correlating this motion data during physical activities or in dynamic environments with the optical signals from the pulse oximeter, the device can differentiate between physiological signals and motion-induced noise, enabling real-time artifact filtering or correction. This ensures more reliable readings, especially in wearable oximeters or when used in conditions involving significant movement, such as during exercise or in medical monitoring of active patients.

Center wavelength drift compensation. We have found that compensating for drift in center wavelength and light output of the red and IR LEDs can improve performance of the photoplethysmogram sensor. LED output covers a spectrum, rather than occurring at a single wavelength, and the shape of that spectrum changes over time. In accordance with a further embodiment of the present invention, in a calibration process, ahead of the actual measurement, the photodetector 208 monitors outputs of the red and IR LEDs and is used by the control circuit to adjust these outputs to reduce the effects of changes in the spectrum of the outputs of the red and IR LEDs.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. An improved photoplethysmogram sensor for measuring a set of parameters related to changes in arterial blood volume in tissue of a subject, the photoplethysmogram sensor being of the type including first and second primary optical sources configured to transmit light into the tissue at selected red and infrared wavelengths respectively, a set of primary light-sensitive semiconductors to measure light returned by the tissue in response to such transmission, and a control circuit coupled to the light sources and the set of primary light-sensitive semiconductors, wherein the improvement comprises:
a set of compensation optical sources configured to illuminate the tissue in the course of a compensation process;
a set of compensation photodetectors configured to measure light, returned by the tissue from the set of compensation optical sources during the compensation process, to provide as an output a signal indicative of the spectral response of the tissue;
wherein the control circuit is coupled to the set of compensation optical sources and to the set of photodetectors and is configured to use the output from the set of photodetectors to compute and to deliver an adjustment to power supplied to the first primary optical source in relation to power supplied to the second primary optical source to compensate for the spectral response of the tissue, in a manner to reduce errors associated with subject-to-subject variation in light transmissivity of tissue.

2. An improved photoplethysmogram sensor according to claim 1, wherein the improvement further comprises an inertial measurement unit (IMU) coupled to the control circuit, and the control circuit is configured to compensate for artifacts attributable to motion of a set of components of the photoplethysmogram sensor.

3. An improved photoplethysmogram sensor according to claim 1, wherein the control circuit is further configured to implement a calibration process, ahead of an actual measurement, in which the set of photodetectors monitors outputs of the first and second primary optical sources and is used by the control circuit to adjust these outputs to reduce the effects of spectral changes in outputs of the first and second primary optical sources.

4. An improved photoplethysmogram sensor according to claim 1, wherein the set of compensation optical sources includes a white-light emitting light source.

5. An improved photoplethysmogram sensor according to claim 1, wherein the set of compensation optical sources includes an RGB photodiode array.

6. An improved photoplethysmogram sensor according to claim 5, wherein the set of compensation optical sources includes an RGB photodiode array.

7. An improved photoplethysmogram sensor according to claim 1, wherein the control circuit is configured to cause carrying out of the compensation process in a distinct compensation time slot of a measurement cycle.

8. An improved photoplethysmogram sensor according to claim 6, wherein the control circuit is configured to cause carrying out of the compensation process in a distinct compensation time slot of a measurement cycle.

9. An improved photoplethysmogram sensor according to claim 7, wherein the control circuit is configured to cause carrying out of the compensation process in a distinct compensation time slot of a measurement cycle.

10. An improved photoplethysmogram sensor according to claim 8, wherein the control circuit is configured to cause carrying out of a data acquisition process, during which the first and second primary optical sources and the primary light-sensitive semiconductors are operational in an acquisition time slot following the compensation time slot in the measurement cycle and the first and second primary optical sources are powered in a manner reflecting the adjustment.

11. An improved photoplethysmogram sensor according to claim 1, wherein the control circuit is configured to use the output from the set of photodetectors to produce an objective measure of the spectral response of the tissue.

12. An improved photoplethysmogram sensor according to claim 11, wherein the control circuit is configured to use the output from the set of photodetectors to determine a value that is correlated with an Individual Typology Angle (ITA) of the tissue.

13. An improved photoplethysmogram sensor according to claim 12, wherein the control circuit is configured to determine the value correlated with the ITA based a set of parameters corresponding to coordinates under the CIE 1931 XYZ standard and thereafter to use such parameters to calculate values corresponding to CIELAB standards based on the CIE 1976 L*a*b* color space.

14. An improved photoplethysmogram sensor for measuring a set of parameters related to changes in arterial blood volume in tissue of a subject, the photoplethysmogram sensor being of the type including first and second primary optical sources configured to transmit light into the tissue at selected red and infrared wavelengths respectively, a set of primary light-sensitive semiconductors to measure light returned by the tissue in response to such transmission, and a control circuit coupled to the light sources and the set of primary light-sensitive semiconductors, wherein the improvement comprises:
a set of compensation optical sources configured to illuminate the tissue in the course of a compensation process;
a set of compensation photodetectors configured to measure light, returned by the tissue from the set of compensation optical sources during the compensation process, to provide as an output a signal indicative of the spectral response of the tissue; and
an inertial measurement unit (IMU) coupled to the control circuit;
wherein the control circuit is coupled to the set of compensation optical sources, and to the set of photodetectors, and to the IMU, and is configured (i) to use the output from the set of photodetectors to compute and to deliver an adjustment to power supplied to the first primary optical source in relation to power supplied to the second primary optical source to compensate for the spectral response of the tissue, in a manner to reduce errors associated with subject-to-subject variation in light transmissivity of tissue, (ii) to compensate for artifacts attributable to motion of a set of components of the photoplethysmogram sensor, and (iii) to implement a calibration process, ahead of an actual measurement, in which the set of photodetectors monitors outputs of the first and second primary optical sources and is used by the control circuit to adjust these outputs to reduce the effects of spectral changes in outputs of the first and second primary optical sources.
